(19) United States
(12) Patent Application Publication
(10) Pub. No.: US 2025/0222236 A1
(43) Pub. Date: Jul. 10, 2025

(54) BACKFLOW INHIBITION APPARATUS

(71) Applicant: TERUMO KABUSHIKI KAISHA, Tokyo (JP)

(72) Inventors: Takito INUKAI, Isehara-shi (JP); Yoichiro KUWANO, Princeton, NJ (US); Takumi FUKUDA, Hadano-shi (JP); Kazuki NAKASONE, Fujinomiya-shi (JP)

(73) Assignee: TERUMO KABUSHIKI KAISHA, Tokyo (JP)

(21) Appl. No.: 19/091,548

(22) Filed: Mar. 26, 2025

Related U.S. Application Data
(63) Continuation of application No. PCT/JP2023/034584, filed on Sep. 22, 2023.

(30) Foreign Application Priority Data
Sep. 26, 2022 (JP) 2022-152986

(51) Int. Cl. A61M 25/06; A61B 8/08; A61M 25/09

(52) U.S. Cl. CPC A61M 25/0693; A61B 8/0891; A61M 25/09041; A61M 2205/3306; A61M 2205/3334; A61M 2205/3375; A61M 2205/581; A61M 2205/583

(57) ABSTRACT

A backflow inhibition apparatus that detects a state of backflow and that performs control for reducing the amount of backflow caused by puncturing a living body using a puncture device includes a backflow detection unit that detects an amount or a rate of backflow from a blood vessel to a proximal end side of the puncture device, a backflow inhibition unit that operates in such a way as to inhibit the amount of backflow into the puncture device, and a control unit that controls operation of the backflow inhibition unit on a basis of the amount or rate of backflow detected by the backflow detection unit, in which the control unit operates the backflow inhibition unit in such a way as to inhibit the amount of backflow if the amount or rate of backflow detected by the backflow detection unit is larger than a first threshold.

### CROSS-REFERENCES TO RELATED APPLICATIONS

This application is a continuation of International Patent Application No. PCT/JP2023/034584 filed on Sep. 22, 2023, which claims priority to Japanese Patent Application No. 2022-152986 filed on Sep. 26, 2022, the entire content of both of which is incorporated herein by reference.

### TECHNOLOGICAL FIELD

The present invention generally relates to a backflow inhibition apparatus that inhibits backflow when a blood vessel is punctured with a needle.

### BACKGROUND DISCUSSION

In order to secure an access site for drug administration or endovascular treatment, vascular puncture for puncturing a human body with an injection needle is performed. Since an operator cannot visually observe a blood vessel from a skin surface as a part of the vascular puncture operation, the operator estimates a position of the blood vessel on the basis of standard knowledge of how blood vessels run and skills including tactile perception of vascular pulsation. The vascular puncture, however, often fails, causing physical and mental distress to patients.

In order to identify a puncture position, techniques for visualizing a position of a blood vessel, such as near-infrared imaging, ultrasound echography, and photoacoustic imaging, have been used these years. By visualizing a position of a blood vessel, the operator can easily determine a puncture position, a puncture angle, a puncture depth, and the like. An apparatus that drives a needle on the basis of obtained information regarding a position of a blood vessel and that automatically performs puncture is also known. An example of an apparatus that automatically performs puncture is the apparatus described in U.S. Pat. No. 9,364,171.

### SUMMARY

When a blood vessel is punctured with a needle as a puncture device, backflow is caused, in which blood flows toward a proximal end of the needle through a lumen of the needle. In order to reduce a burden on a body, it is desirable to keep the backflow as small as possible. When the operator manually performs puncture, the amount of backflow is reduced by appropriately pressing a proximal end of a puncture device with a hand or the like while checking a state of the backflow. In an apparatus that automatically performs puncture, control for reducing the amount of backflow has not been performed.

Disclosed here is a backflow inhibition apparatus that detects a state of backflow and that performs controls for reducing the amount of backflow.

A backflow inhibition apparatus according to one aspect of the disclosure here includes a puncture device, a puncture drive unit that punctures a living body with the puncture device, a backflow detection unit configured to detect an amount or a rate of backflow from a blood vessel to a proximal end side of the puncture device, a backflow inhibition unit configured to inhibit the amount of backflow into the puncture device, and a control unit configured to drive the puncture drive unit and to control a position of the puncture device, in which the control unit operates the backflow inhibition unit in such a way as to inhibit the amount of backflow if the amount or rate of backflow detected by the backflow detection unit is larger than a threshold while the puncture drive unit is inserting the puncture device.

Since the backflow inhibition apparatus configured as described above compares the amount or rate of backflow detected by the backflow detection unit with the threshold and then operates the backflow inhibition unit in such a way as to inhibit the amount of backflow if the amount of backflow is large, it is possible to inhibit an outflow of the backflow to reduce a burden on a body and to keep the outflow from becoming an obstacle when treatment proceeds.

According to another aspect, a vascular puncture apparatus for puncturing a living body comprises: a main body positionable adjacent the living body; a puncture device held by the main body and movable towards the living body to cause a distal end of the puncture device to puncture the living body and enter a blood vessel in the living body, with the puncture device comprising a hollow needle and a hub on a proximal end side of the hollow needle; a camera or sensor provided on the puncture device to detect blood backflow from the blood vessel after the puncture device punctures the living body; and a backflow inhibition unit configured to inhibit an amount of the blood backflow into the puncture device. A control unit is operatively connected to: i) the camera or sensor provided on the puncture device to receive information from the camera or about the amount or rate of the blood backflow from the blood vessel after the puncture device punctures the living body; and ii) the backflow inhibition unit to control operation of the backflow inhibition unit based on the information received from the camera or sensor about the amount or rate of the blood backflow detected by the backflow detection unit. The control unit is configured to control operation of the backflow inhibition unit to inhibit the amount of the blood backflow if the amount or rate of the blood backflow detected by the backflow detection unit is larger than a threshold.

Another aspect involves a method comprising: advancing a puncture device toward a living body to puncture the living body with the puncture device and cause a distal end of the puncture device to enter a blood vessel in the living body, with the distal end of the puncture device entering the blood vessel in the living body resulting in blood backflow from the blood vessel to a proximal end side of the puncture device; detecting an amount or rate of the blood backflow from the blood vessel to the proximal end side of the puncture device; and reducing or stopping the amount or rate of the blood backflow when the amount or rate of blood backflow exceeds a threshold.

### BRIEF DESCRIPTION OF THE DRAWINGS

FIG. 1 is a front view of a vascular puncture apparatus according to an embodiment.

FIG. 2 is a perspective view illustrating an internal structure of a puncture drive unit and a guide wire drive device.

FIG. 3 is a cross-sectional view of the vicinity of a backflow inhibition unit of a puncture device.

FIGS. 4a and 4b are cross-sectional views of the vicinity of a backflow inhibition unit according to a modification of the puncture device, where FIG. 4a is a view illustrating a state in which backflow is not inhibited, and FIG. 4b is a view illustrating a state in which backflow is inhibited.

FIG. 5 is a configuration diagram of the vascular puncture apparatus.

FIG. 6 is a diagram illustrating a skin contact surface of a probe body and is a diagram illustrating a positional relationship with an arm for which a cross-sectional image is to be obtained.

FIG. 7 is a flowchart of puncture using the vascular puncture apparatus.

FIG. 8 is a conceptual diagram of an obtained echogram.

FIG. 9 is a cross-sectional view illustrating a positional relationship between a blood vessel and an imaging unit.

FIGS. 10a-10c are diagrams illustrating a relationship between an inner needle and the blood vessel during insertion.

FIG. 11 is a flowchart according to a first modification of the puncture using the vascular puncture apparatus.

FIG. 12 is a flowchart according to a second modification of the puncture using the vascular puncture apparatus.

FIG. 13 is a flowchart according to a third modification of the puncture using the vascular puncture apparatus.

FIGS. 14a-14e are diagrams illustrating a relationship between a puncture device equipped with an outer tube and the blood vessel.

FIG. 15 is a flowchart according to a fourth modification of the puncture using the vascular puncture apparatus.

FIGS. 16a and 16b are diagrams illustrating a relationship between the puncture device and the blood vessel, where FIG. 16a is a diagram illustrating a state in which the inner needle is located inside the blood vessel and the outer tube is located outside the blood vessel, and FIG. 16b is a diagram illustrating a state in which the inner needle is located outside the blood vessel and the outer tube is located inside the blood vessel.

FIG. 17 is a front view of a vascular puncture apparatus according to a modification.

### DETAILED DESCRIPTION

Embodiments of the backflow inhibition apparatus representing examples of the backflow inhibition apparatus disclosed here will be described hereinafter with reference to the drawings. Dimensional ratios in the drawings might be exaggerated for convenience of description and differ from actual ratios.

A vascular puncture apparatus 10 according to one embodiment is used when puncturing an arm of a human body, and obtains a cross-sectional image of the arm, detects a position of a blood vessel, determines a puncture timing and puncture parameters, and punctures the blood vessel with a needle.

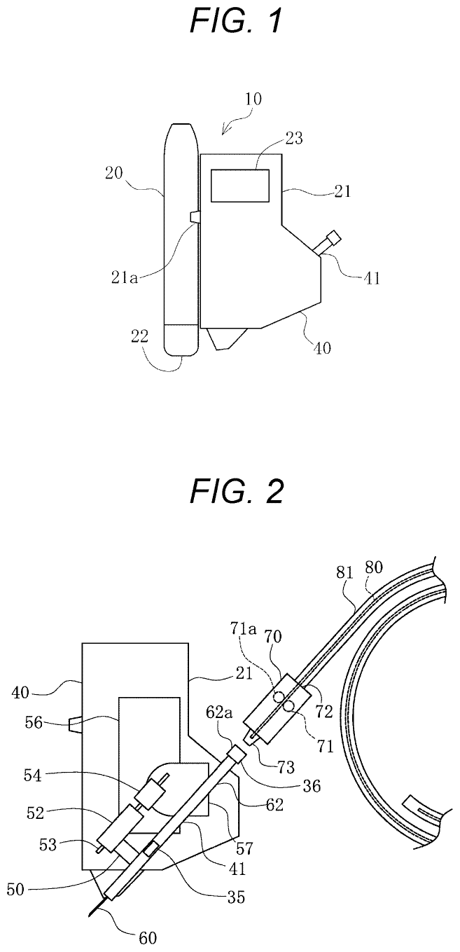

As illustrated in FIG. 1, the vascular puncture apparatus 10 including a backflow inhibition apparatus includes a probe body 20 including an imaging unit 22 that comes into contact with a skin surface and that obtains a cross-sectional image of a human body, and a puncture drive unit 21 that holds a puncture device 41 and that performs puncture by driving the puncture device. The puncture drive unit 21 has a fixing portion 21a engaged with and fixed to the probe body 20, and the probe body 20 and the puncture drive unit 21 are integrated together.

As illustrated in FIG. 2, the vascular puncture apparatus 10 includes the puncture drive unit 21 that moves the puncture device 41 and a guide wire drive device 70 that inserts a guide wire 80 into the puncture device 41.

The puncture drive unit 21 includes a main body 40 that holds the puncture device 41. The main body 40 includes a notification unit 23 for notifying an operator of a puncture state and the like. The notification unit 23 can be configured by a display or the like that displays information. The notification unit 23, however, may be a simpler display unit such as an LED, instead. Alternatively, the notification unit 23 may perform the notification using sound.

The puncture device 41 includes an inner needle 60 that is a hollow needle and that punctures a blood vessel and a hub 62 provided on a proximal end side of the inner needle 60. The inner needle 60 has a distal end opened, and a proximal end thereof communicates with a lumen of the hub 62. The hub 62 includes, at a proximal end thereof, an insertion portion 62a protruding from the main body 40. The insertion portion 62a communicates with the lumen of the hub 62, and a guide wire 80 can be inserted. The guide wire 80 inserted from the insertion portion 62a can be inserted into the inner needle 60 toward a distal end of the inner needle 60 through the lumen of the hub 62 and the inside of the inner needle 60.

The hub 62 is provided with a backflow detection unit 35 that detects backflow from a blood vessel toward a proximal end of the inner needle 60. The backflow detection unit 35 is a sensor capable of detecting a flow of blood in the lumen of the hub 62 from a distal end toward the proximal end of the hub 62, and can be configured by a camera. The backflow detection unit 35 can be configured by a pressure sensor, a proximity sensor, or the like instead of a camera. In addition, the backflow detection unit 35 may be fixed to the main body 40.

The backflow detection unit 35 has a configuration capable of detecting the amount or rate of backflow. For this purpose, the hub 62 is provided with a measuring tube with a scale branching from the lumen, and the backflow detection unit 35 including a camera can detect the amount of backflow by detecting a division at which blood flowing into the measuring tube reaches. When the backflow detection unit 35 is configured by cameras, a plurality of cameras may be arranged in a lumen 62b of the hub 62 in a longitudinal direction of the lumen 62b, and a rate of backflow can be detected from a rate at which blood passes between two points. Alternatively, the backflow detection unit 35 may include a flow meter in the lumen of the hub 62.

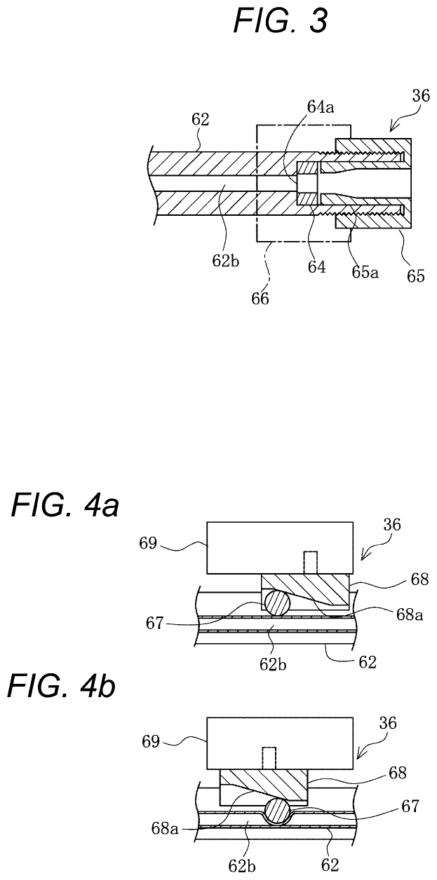

The hub 62 includes a backflow inhibition unit 36 that stops backflow or reduces the amount of backflow. As illustrated in FIG. 3, the backflow inhibition unit 36 includes a valve body 64 having an opening 64a disposed in the lumen 62b of the hub 62, and a pressing body 65a disposed in such a way as to face the valve body 64. The pressing body 65a is provided in a cap 65 disposed at the proximal end of the hub 62. The cap 65 also functions as the insertion portion 62a of the hub 62.

The cap 65 is screwed to an outer peripheral surface of the hub 62, and can move forward and backward with respect to the hub 62 through rotation. By rotating the cap 65, the pressing body 65a moves toward the valve body 64, and the valve body 64 is pressed in the longitudinal direction of the lumen 62b. When the valve body 64 is pressed by the pressing body 65a, the opening 64a is blocked, and the amount of backflow through the lumen 62b of the hub 62 can be reduced or the backflow can be stopped. The cap 65 is automatically rotated by a rotation drive unit 66.

As illustrated in FIG. 4a, the backflow inhibition unit 36 may include a ball-shaped pressing body 67 disposed adjacent to the lumen 62b of the hub 62, a pressing drive body 68 that moves the pressing body 67 toward the lumen 62b, and a pressing drive portion 69 that horizontally moves the pressing drive body 68. The pressing drive body 68 has an inclined surface 68a. As illustrated in FIG. 4b, the pressing drive portion 69 horizontally moves the pressing drive body 68, so that the pressing body 67 is pressed toward the lumen 62b due to the inclined surface 68a. Since the lumen 62b of the hub 62 is configured by the tube 62c having flexibility, the lumen 62b is collapsed when pressed by the pressing body 67, and it is possible to reduce the amount of backflow through the lumen 62b or stop the backflow.

The puncture drive unit 21 includes a horizontal direction drive unit 56 supported by the main body 40, a biaxial drive unit 57 supported by the horizontal direction drive unit 56, a puncture direction drive unit 54 supported by the biaxial drive unit 57, a puncture direction guide unit 53 supported by the puncture direction drive unit 54, and a puncture direction movement unit 52 supported by the puncture direction guide unit 53 and movable in a puncture direction. FIG. 2 illustrates X, Y, and Z directions. The X direction corresponds to a width direction of the arm, the Y direction corresponds to a depth direction of the arm, and the Z direction corresponds to a length direction of the arm. The puncture device 41 is fixed to a holding unit 50 provided for the puncture direction movement unit 52.

The biaxial drive unit 57 allows the puncture device 41 to change orientation thereof relative to an X-Z plane. As a result, the orientation of the puncture device 41 with respect to a planar direction of the skin surface can be changed, and the puncture device 41 can enter a blood vessel 100 straight. The horizontal direction drive unit 56 allows the puncture device 41 to move in the X direction. As a result, the puncture device 41 can be moved in the planar direction of the skin surface, and the puncture device 41 can be arranged in such a way as to be able to enter the blood vessel 100. The biaxial drive unit 57 allows the puncture device 41 to change inclination thereof in a Y-Z plane. As a result, an entry angle of the puncture device 41 with respect to the skin surface can be changed.

The guide wire drive device 70 includes a guide wire insertion unit 72 in which an end of a holder tube 81 holding the guide wire 80 is held and into which the guide wire 80 is inserted, a guide wire drive unit 71 that moves the inserted guide wire 80, and a coupling unit 73 coupled to the insertion portion 62a of the hub 62 of the puncture device 41. The guide wire drive unit 71 includes two rollers 71a sandwiching the guide wire 80, and can move the guide wire 80 to both sides in a longitudinal direction.

The guide wire drive device 70 is coupled to the hub 62 after the puncture drive unit 21 causes the puncture device 41 to puncture the blood vessel. The coupling of the guide wire drive device 70 to the hub 62 is performed by the operator, but a coupling device that moves the guide wire drive device 70 and couples the guide wire drive device to the hub 62 may also be provided. This allows the guide wire drive device 70 to be automatically coupled to the puncture device 41.

The guide wire drive device 70 can also be used as the backflow inhibition unit 36. By inserting the guide wire 80 into the lumen of the hub 62, the lumen is blocked to some extent, so that the amount of backflow can be reduced.

As illustrated in FIG. 5, the vascular puncture apparatus 10 includes an imaging unit 22 that comes into contact with the skin surface and that obtains a cross-sectional image of the human body and a control unit 30 that detects a position of a blood vessel from the cross-sectional image and that controls a position of the puncture device 41 by driving the puncture drive unit 21 and the guide wire drive unit 71. The control unit 30 is connected to the imaging unit 22 via a transmission unit 32 and a reception unit 34, can cause the imaging unit 22 to obtain a cross-sectional image, and can receive the obtained cross-sectional image. The control unit 30 is connected to the backflow detection unit 35, and can obtain information regarding backflow detected by the backflow detection unit 35. The control unit 30 is also connected to a notification unit 23 provided for the main body 40, and can cause the notification unit 23 to perform notification. In addition, the control unit 30 can drive the backflow inhibition unit 36 to perform control for reduce the amount of backflow or stop the backflow. In the vascular puncture apparatus 10, the backflow inhibition apparatus that inhibits the amount of backflow can be configured by at least the control unit 30, the backflow detection unit 35, and the backflow inhibition unit 36. In addition, the backflow inhibition apparatus can include the imaging unit 22, the transmission unit 32, and the reception unit 34.

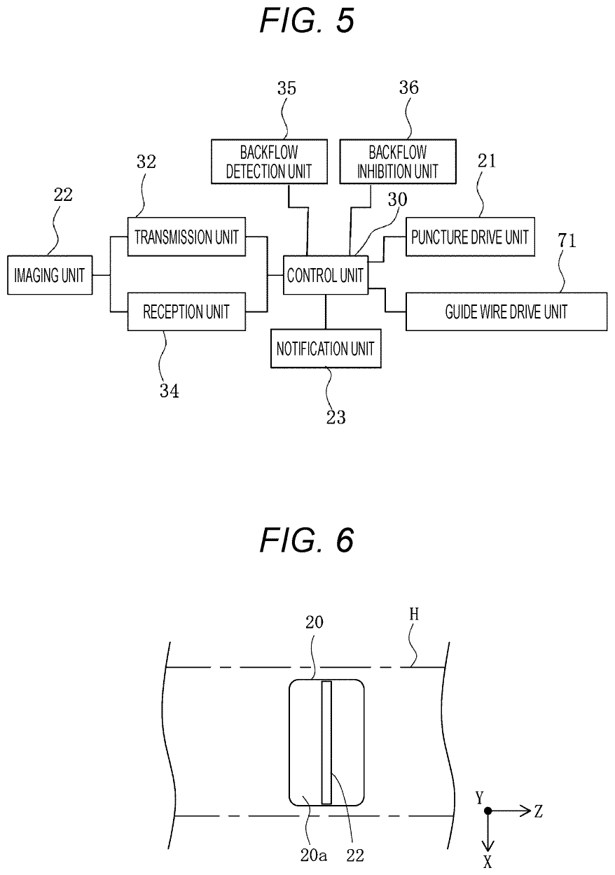

As illustrated in FIG. 6, the imaging unit 22 of the probe body 20 is provided in such a way as to extend, in one direction, over substantially entire width of a skin contact surface 20a of the probe body 20 at a central portion of the skin contact surface 20a. The imaging unit 22 is an echograph that includes a transducer which generates an ultrasonic wave and that obtains a cross-sectional image of the inside of the human body by detecting the reflected ultrasonic wave. In the present embodiment, since the imaging unit 22 obtains a cross-sectional image orthogonal to an axial direction of the blood vessel, the imaging unit 22 is arranged such that a length direction of the imaging unit 22 becomes orthogonal to a length direction of an arm H.

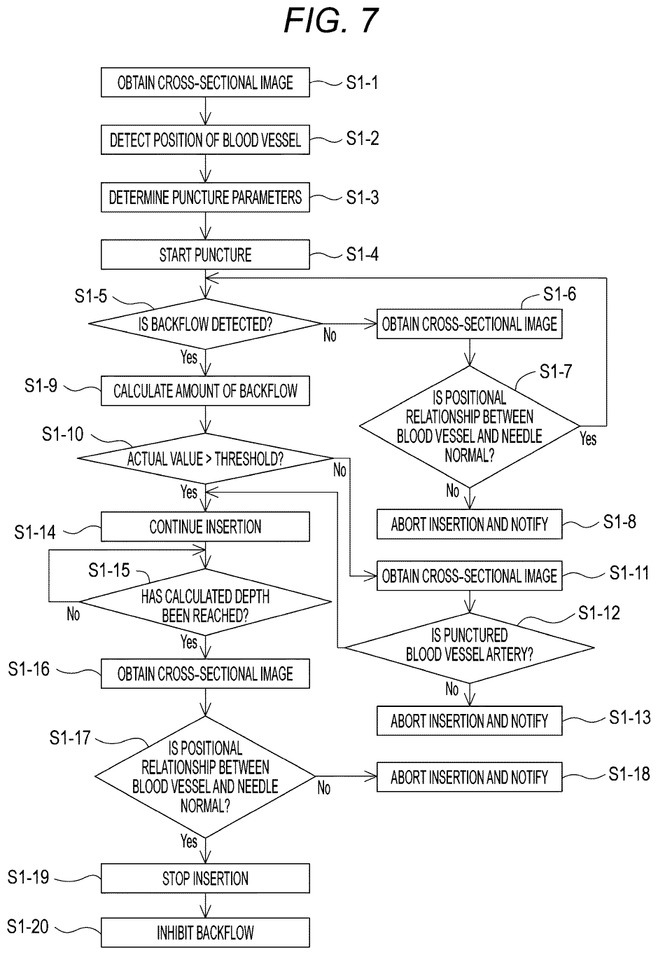

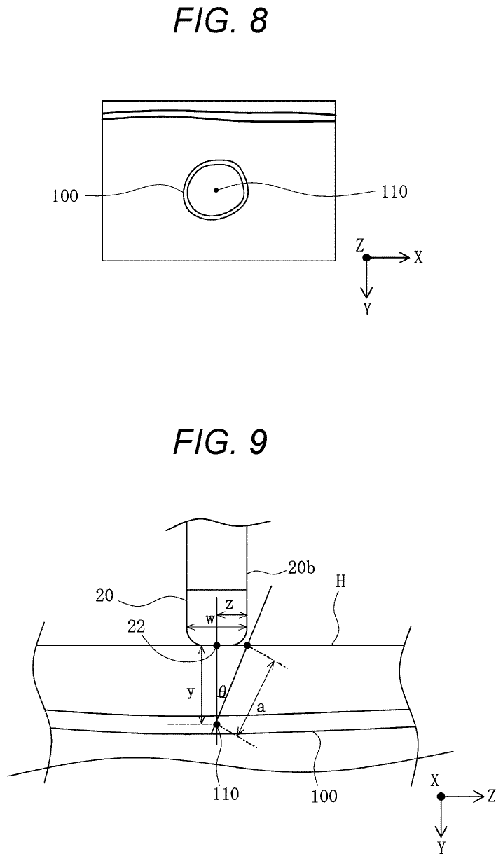

Next, operation of the vascular puncture apparatus 10 will be described as a part of a puncture procedure. As illustrated in FIG. 7, the control unit 30 obtains a cross-sectional image as illustrated in FIG. 8 from the imaging unit 22 (S1-1). The cross-sectional image includes an image of the blood vessel 100. A horizontal direction in the cross-sectional image is the X direction, a vertical direction in the cross-sectional image is the Y direction, and a direction orthogonal to a paper surface of the cross-sectional image is the Z direction. Coordinates of an upper-left point in the cross-sectional image are defined as a start point (0, 0, 0).

The control unit 30 detects a position of the blood vessel 100 in the obtained cross-sectional image by analyzing the image (S1-2). The control unit 30 detects a region recognized as the blood vessel 100 in the image, and sets a barycentric position 110 of the region as the position of the blood vessel. In order to detect a region recognized as a blood vessel in an image, a large number of images of the same type can be prepared, and a method of machine learning or deep learning can be used. In addition, it is also possible to detect a region with blood flow by a Doppler method in the imaging unit 22 and recognize the region as a region of a blood vessel. The region of the blood vessel needs to be detected from the cross-sectional image while distinguishing an artery and a vein. An artery and a vein can be distinguished on the basis of a position of a bone of the arm H in the cross-sectional image. In addition, when a region with blood flow is detected by the Doppler method, an artery and a vein can be distinguished on the basis of a direction of the blood flow. Coordinates of the detected barycentric position 110 of the blood vessel are defined as (x, y, 0).

Next, the control unit 30 determines parameters necessary for puncture (S1-3). The parameters necessary for puncture include a puncture direction in the X-Z plane, a puncture position, an entry angle of the puncture device 41, an insertion depth of the puncture device 41, and a puncture speed of the puncture device 41. As illustrated in FIG. 7, since the barycentric position 110 of the blood vessel has been detected from the cross-sectional image obtained by the imaging unit 22, the puncture direction in the X-Z plane is determined such that the puncture device 41 of the puncture drive unit 21 fixed to the probe body 20 is directed to the barycentric position 110.

The puncture position is a position immediately below a side surface 20b of the probe body 20. In this case, a coordinate z of the side surface 20b of the probe body 20 in the Z direction, which is the puncture position, is half a width W of the probe body 20, and is therefore calculated as z=W/2. The puncture angle is an angle θ of a line from the barycentric position of the blood vessel to the puncture position relative to a perpendicular line of the skin surface, and is calculated as θ=arctan(z/y). The puncture depth a is calculated as a=y/cos·θ. The coordinate of the puncture position in the x direction and the puncture depth a of the puncture device 41 are thus defined.

As the parameters of puncture, other parameters may also be used. For example, a puncture start time, a puncture end time, puncture acceleration at a start of puncture, puncture acceleration at an end of puncture, and the like can be used.

After determining the parameters of puncture, the control unit 30 operates the puncture drive unit 21 in accordance with the parameters of puncture to start puncture (S1-4).

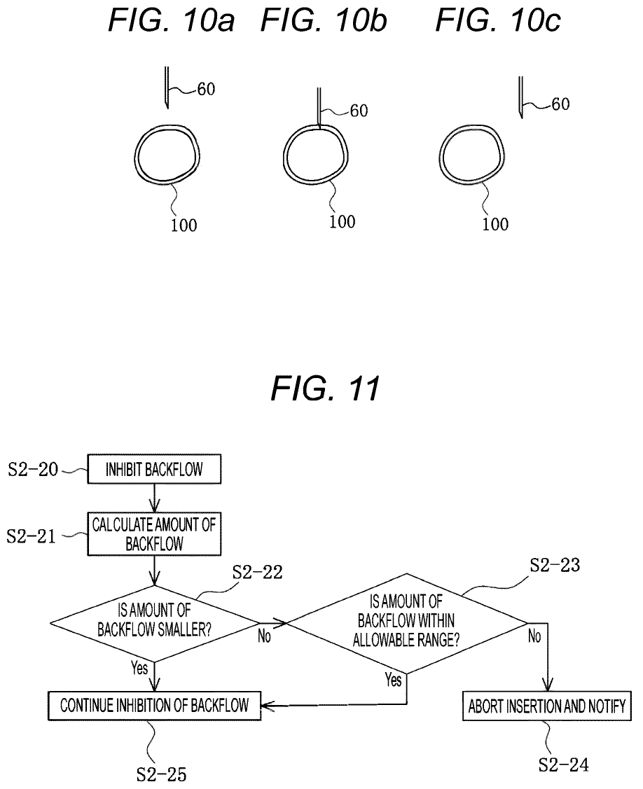

The control unit 30 determines whether or not the backflow detection unit 35 detects backflow when the puncture drive unit 21 is inserting the puncture device 41 (S1-5). If the backflow detection unit 35 does not detect backflow, the control unit 30 obtains a cross-sectional image using the imaging unit 22 (S1-6), and determines, on the basis of the obtained cross-sectional image, whether or not a positional relationship between the inner needle 60 and the blood vessel 100 is normal (S1-7). If the inner needle 60 is directed toward the blood vessel 100 in the obtained cross-sectional image as illustrated in FIGS. 10(a) and 10(b), it is determined that the positional relationship between the inner needle 60 and the blood vessel 100 is normal. If the inner needle 60 is not directed toward the blood vessel 100 as illustrated in FIG. 10c, it is determined that the positional relationship between the inner needle 60 and the blood vessel 100 is not normal. This determination can be performed by detecting, in the obtained cross-sectional image through an image analysis, coordinates of the distal end of the inner needle 60 and a coordinate range in which the blood vessel 100 is present. Alternatively, the positional relationship between the inner needle 60 and the blood vessel 100 may be determined by a method of machine learning or deep learning.

If it is determined in S1-7 that the positional relationship between the inner needle 60 and the blood vessel 100 is normal, the process returns to step S1-5. If it is determined in S1-7 that the positional relationship between the inner needle 60 and the blood vessel 100 is not normal, the control unit 30 aborts the insertion of the puncture device 41, and the notification unit 23 provides a notification of the abortion (S1-8).

When backflow is detected, the control unit 30 calculates the amount of backflow from information regarding the backflow detected by the backflow detection unit 35 (S1-9). After calculating the amount of backflow, the control unit 30 determines whether or not a measured value of the amount of backflow is larger than a threshold (S1-10). The threshold for the amount of backflow is set on the basis of the amount of backflow estimated to occur when the puncture device 41 punctures an artery. As the estimated amount of backflow, a value determined in advance from biological information regarding a puncture subject (patient), such as age, weight, blood vessel diameter, and blood pressure, can be used. In addition, the threshold for the amount of backflow may be calculated as a predicted amount of backflow by obtaining blood pressure and blood vessel diameter from the obtained cross-sectional image. In addition, machine learning may be performed in advance using data regarding a relationship between cross-sectional images and the amount of backflow, and an optimum value as the threshold value may be calculated by applying a model of the machine learning to the obtained cross-sectional image. Although the control unit 30 calculates the amount of backflow in S1-9, the control unit 30 may calculate a rate of backflow, instead.

If the measured value of the backflow is smaller than the threshold in S1-10, the control unit 30 obtains a cross-sectional image using the imaging unit 22 (S1-11), and determines whether or not the punctured blood vessel is an artery from the obtained cross-sectional image (S1-12). Whether or not the punctured blood vessel is an artery can be determined similarly to the determination in S1-2. If the punctured blood vessel is not an artery, the control unit 30 aborts the insertion of the puncture device 41, and the notification unit 23 notifies of the abortion (S1-13). If the punctured blood vessel is an artery, the process proceeds to S1-14.

If the measured value of the backflow is larger than the threshold in S1-10, the control unit 30 continues the insertion of the puncture device 41 (S1-14). The control unit 30 determines whether or not the insertion depth of the puncture device 41 has reached a calculated depth while the puncture device 41 is being inserted (S1-15). If the insertion depth of the puncture device 41 reaches the calculated depth, the control unit 30 obtains a cross-sectional image using the imaging unit 22 (S1-16), and determines, on the basis of the obtained cross-sectional image, whether or not the positional relationship between the inner needle 60 and the blood vessel 100 is normal (S1-17). If the positional relationship between the inner needle 60 and the blood vessel 100 is not normal in S1-17, the control unit 30 performs abortion processing similar to that in S1-13 (i.e., the control unit 30 aborts the insertion of the puncture device 41). If the positional relationship between the inner needle 60 and the blood vessel 100 is normal, the control unit 30 stops the insertion of the puncture device 41 (S1-19).

After stopping the insertion of the puncture device 41, the control unit 30 operates the backflow inhibition unit 36 to inhibit the backflow (S1-20). As a result, the vascular puncture apparatus 10 can inhibit the amount of backflow generated during puncture and reduce a burden on a body. In addition, since the vascular puncture apparatus 10 can inhibit an outflow of backflow, it is possible to keep the outflow from becoming an obstacle when treatment proceeds.

Next, a first modification of the puncture using the vascular puncture apparatus 10 will be described. In this example, since a flow or operation (S1-1 to S1-20) until inhibition of backflow by the backflow inhibition unit 36 is the same as that described above regarding FIG. 7, a detailed description of such operational aspects is not repeated.

In FIG. 11, after starting the inhibition of backflow, the control unit 30 calculates the amount of backflow from information regarding the backflow detected by the backflow detection unit 35 (S2-21). The control unit 30 determines whether or not the calculated amount of backflow is smaller than that before the inhibition of backflow (S2-22). If the amount of backflow is not smaller than that before the inhibition of backflow, the control unit 30 determines whether or not the amount of backflow calculated in S2-21 is within an allowable range (S2-23). The allowable range of the amount of backflow can be set in advance similarly to the threshold in S1-10. If the amount of backflow is within the allowable range, the control unit 30 continues the inhibition of backflow (S2-25). If the amount of backflow is not within the allowable range, the control unit 30 performs abortion processing similar to that in S1-13 (i.e., the control unit 30 aborts the insertion of the puncture device 41) and provides notification of such aborted insertion of the puncture device). If the amount of backflow is smaller in S2-22, the control unit 30 continues the inhibition of backflow (S2-25). In this example, too, the backflow detection unit 35 may detect the rate of backflow, and the control unit 30 may perform control on the basis of the rate of backflow.

Since the control unit 30 determines whether or not the amount of backflow has decreased after a start of the inhibition of backflow, it is thus possible to confirm whether or not the backflow inhibition unit 36 is sufficiently functioning and to perform reliable inhibition of backflow.

Next, a second modification of the puncture using the vascular puncture apparatus 10 will be described. In this example, a flow or operation (S1-1 to S1-19) until the puncture device 41 punctures the blood vessel to stop the insertion is the same as that in FIG. 7, and so a detailed description of such operational aspects is not repeated.

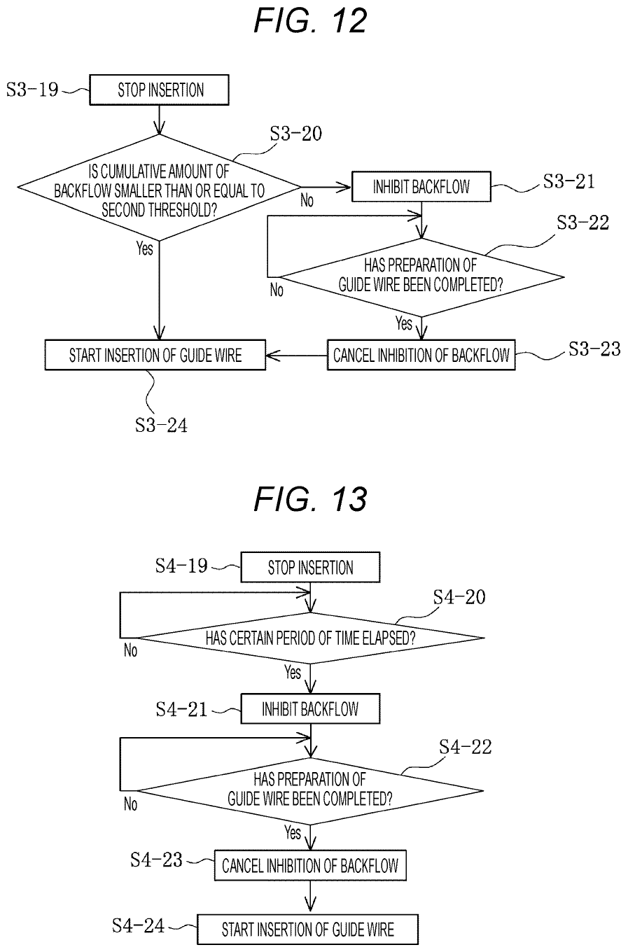

In FIG. 12, the control unit 30 calculates a cumulative amount of backflow after the backflow detection unit 35 first detects backflow. Then, after stopping the insertion of the puncture device 41 (S3-19), the control unit 30 determines whether or not the calculated cumulative amount of backflow is smaller than or equal to a second threshold (S3-20). If the cumulative amount of backflow is larger than the second threshold, the control unit 30 operates the backflow inhibition unit 36 to inhibit the amount of backflow (S3-21). Next, the control unit 30 determines whether or not preparation for inserting the guide wire 80 from the hub 62 has been completed (S3-22). Whether or not the preparation of the guide wire 80 has been completed can be determined by detecting coupling of the coupling unit 73 of the guide wire drive device 70 to the insertion portion 62a of the hub 62. In addition, when the control unit 30 controls the movement of the guide wire drive device 70, the control unit 30 can detect the completion of the preparation of the guide wire 80 by coupling the guide wire drive device 70 to the coupling unit 73.

If the control unit 30 detects the completion of the preparation of the guide wire 80, the control unit 30 cancels the inhibition of backflow by the backflow inhibition unit 36 (S3-23). As a result, the lumen 62b of the hub 62 communicates with the inner needle 60, and the guide wire 80 can be inserted into the blood vessel via the inner needle 60. After canceling the inhibition of backflow, the control unit 30 starts the insertion of the guide wire 80 using the guide wire drive unit 71 (S3-24). If the cumulative amount of backflow is smaller than or equal to the second threshold in S3-20, the control unit 30 starts the insertion of the guide wire 80 using the guide wire drive unit 71 without inhibiting the backflow (S3-24).

The vascular puncture apparatus 10 can thus further reduce the burden on the body by determining whether or not to perform the inhibition of backflow on the basis of the cumulative amount of backflow. In addition, since the vascular puncture apparatus 10 cancels the inhibition of backflow after the preparation of the guide wire is completed, it is possible to prevent the insertion of the guide wire 80 from being hindered.

The control unit 30 may determine whether or not the calculated cumulative amount of backflow is smaller than or equal to the second threshold when the actual insertion depth of the puncture device 41 reaches an estimated insertion depth. If the cumulative amount of backflow is larger than the second threshold, the control unit 30 operates the backflow inhibition unit 36 to inhibit the amount of backflow.

Next, a third modification of the puncture using the vascular puncture apparatus 10 will be described. In this example, a flow or operation (S1-1 to S1-19) until the puncture device 41 punctures the blood vessel to stop the insertion is the same as that in FIG. 7, and so a detailed description of such operational aspects is not repeated.

In FIG. 13, the control unit 30 measures time elapsed since the backflow detection unit 35 first detected backflow. Then, after stopping the insertion of the puncture device 41 (S4-19), the control unit 30 determines whether or not a certain period of time has elapsed since backflow was first detected (S4-20). If the certain period of time has elapsed since backflow was first detected, the control unit 30 operates the backflow inhibition unit 36 to inhibit the amount of backflow (S4-21). Next, the control unit 30 determines whether or not the preparation for inserting the guide wire 80 from the hub 62 has been completed (S4-22), and if the preparation of the guide wire 80 has been completed, cancels the inhibition of backflow by the backflow inhibition unit 36 (S4-23). Thereafter, the control unit 30 starts the insertion of the guide wire 80 using the guide wire drive unit 71 (S4-24).

The vascular puncture apparatus 10 can thus further reduce the burden on the body by starting the inhibition of backflow when the certain period of time has elapsed since backflow was first detected.

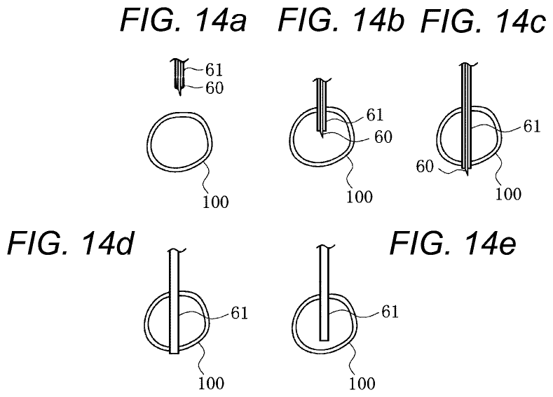

Next, a fourth modification of the puncture using the vascular puncture apparatus 10 will be described. In this example, as illustrated in FIG. 14a, the puncture device 41 includes an inner needle 60 and an outer tube 61 covering the inner needle 60, and a distal end of the inner needle 60 protrudes from a distal end of the outer tube 61 (the inner needle 60 protrudes on the distal side of the distal end of the outer tube 61). In addition, the inner needle 60 can be moved to a proximal end of the outer tube 61 by the puncture drive unit 21 and removed. The backflow detection unit 35 detects backflow between the inner needle 60 and the outer tube 61.

When the puncture device 41 provided with the outer tube 61 is inserted into the blood vessel 100, both the inner needle 60 and the outer tube 61 enter the blood vessel as illustrated in FIG. 14b. At this time, backflow occurs in the inner needle 60 and between the inner needle 60 and the outer tube 61.

When the puncture device 41 is further inserted, both the inner needle 60 and the outer tube 61 penetrate vessel walls of the blood vessel on a side of the insertion and an opposite side as illustrated in FIG. 14c. At this time, since the distal end of the inner needle 60 and a distal end of the outer tube 61 are located outside the blood vessel, backflow does not occur.

As illustrated in FIG. 14d, when the inner needle 60 and the outer tube 61 penetrate the blood vessel, the inner needle 60 is removed. Furthermore, as illustrated in FIG. 14e, by moving the remaining outer tube 61 to a proximal end side (in a proximal side direction), the distal end of the outer tube 61 is located in the blood vessel, and the guide wire 80 can be inserted.

A flow of the puncture operation in this example will be described. Because a flow or operation (S1-1 to S1-3) until the puncture parameters are determined in this example is the same as that in FIG. 7, a detailed description of such operational aspects is not repeated.

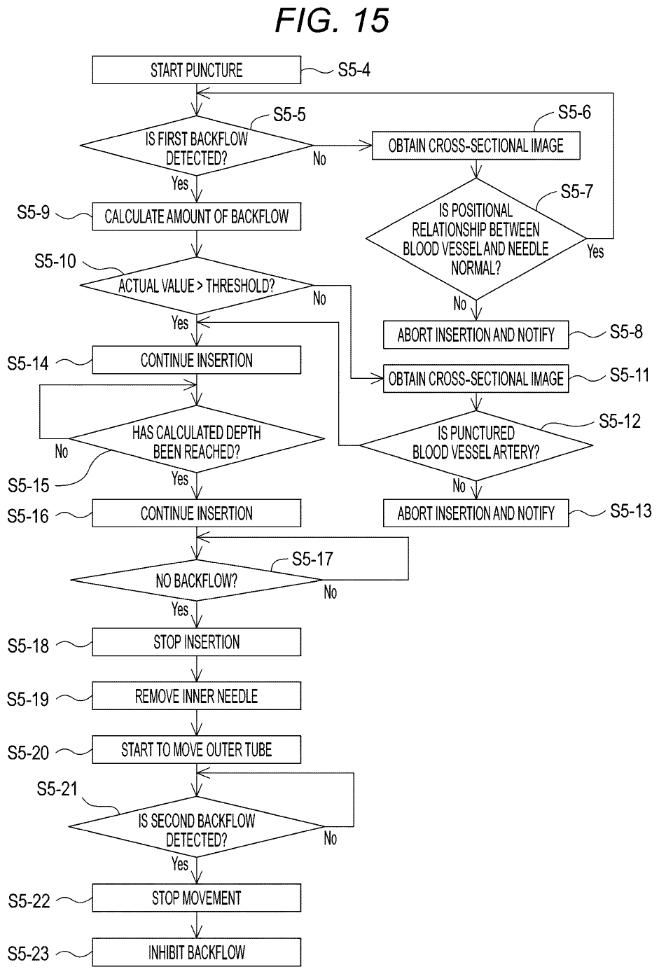

In FIG. 15, when the puncture of the puncture device 41 is started (S5-4), the control unit 30 determines whether or not the backflow detection unit 35 detects a first backflow while the puncture drive unit 21 is inserting the puncture device 41 (S5-5). The first backflow is a backflow that first occurs after the puncture device 41 starts puncture, and is a backflow that occurs in a state of FIG. 14b. If the backflow detection unit 35 does not detect backflow, the control unit 30 obtains a cross-sectional image using the imaging unit 22 (S5-6), and determines, on the basis of the obtained cross-sectional image, whether or not the positional relationship between the inner needle 60 and the blood vessel 100 is normal (S5-7). If the positional relationship between the inner needle 60 and the blood vessel 100 is not normal, the control unit 30 aborts the insertion of the puncture device 41, and the notification unit 23 provides a notification that the puncture device insertion has been aborted (S5-8).

If the backflow detection unit 35 detects the first backflow in S5-5, the control unit 30 calculates the amount of backflow from information regarding the backflow detected by the backflow detection unit 35 (S5-9). After calculating the amount of backflow, the control unit 30 determines whether or not a measured value of the amount of backflow is larger than a threshold (S5-10).

If the measured value of the backflow is smaller than the threshold in S5-10, the control unit 30 obtains a cross-sectional image using the imaging unit 22 (S5-11), and determines whether or not the punctured blood vessel is an artery from the obtained cross-sectional image (S5-12). Whether or not the punctured blood vessel is an artery can be determined similarly to the determination in S1-2. If the punctured blood vessel is not an artery, the control unit 30 aborts the insertion of the puncture device 41, and the notification unit 23 notifies of the abortion (S5-13). If the punctured blood vessel is an artery, the process proceeds to S5-14.

If the measured value of the backflow is larger than the threshold in S5-10, the control unit 30 continues the insertion of the puncture device 41 (S5-14). The control unit 30 determines whether or not the insertion depth of the puncture device 41 has reached a calculated depth while the puncture device 41 is being inserted (S5-15). Here, since the puncture device 41 is once inserted to a position penetrating the blood vessel as illustrated in FIG. 14c, when the insertion depth of the puncture device 41 reaches the calculated depth, the control unit 30 further continues the insertion of the puncture device 41 (S5-16).

The control unit 30 continues the insertion of the puncture device 41 until the backflow detection unit 35 no longer detects the backflow (S5-17). When the backflow of the puncture device 41 is no longer detected, the control unit 30 stops the insertion while assuming that the puncture device 41 has penetrated the blood vessel (S5-18).

Next, the control unit 30 removes the inner needle 60 from the outer tube 61 (S5-19). Subsequently, the control unit 30 starts to move the outer tube 61 to the proximal end side or proximal side direction (S5-20). The control unit 30 determines whether or not the backflow detection unit 35 detects a second backflow while the outer tube 61 is being moved (S5-21). The second backflow is a backflow that occurs again after backflow stops once, and is a backflow that occurs in a state of FIG. 14e. The second backflow has a larger amount or a higher rate than the first backflow, since the inner needle 60 has been removed.

If the backflow detection unit 35 detects the second backflow having a larger amount or a higher rate than the first backflow in S5-21, the control unit 30 stops the movement of the outer tube 61 (S5-22) and operates the backflow inhibition unit 36 to inhibit the backflow (S5-23). The flows or operations shown in FIGS. 11 to 13 may be further added to the flow in the present example.

As described above, the vascular puncture apparatus 10 including the puncture device 41 provided with the outer tube 61 can also perform control of the puncture device 41 and control of the inhibition of backflow on the basis of information regarding the backflow.

The backflow detection unit 35 may be configured to be able to detect backflow into the proximal end side of the inner needle 60 in addition to backflow between the inner needle 60 and the outer tube 61. In this case, the backflow detection unit 35 includes a sensor such as a camera in each of the outer tube 61 and the inner needle 60 or a tubular body communicating with these.

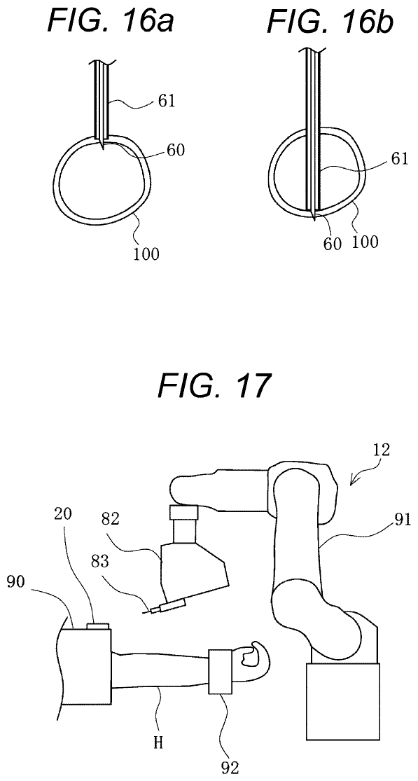

As illustrated in FIG. 16a, in a state in which only the inner needle 60 protruding from the outer tube 61 has entered the blood vessel 100 but the outer tube 61 has not reached the blood vessel (interior of the blood vessel) 100, backflow into the proximal end side of the inner needle 60 occurs, but backflow does not occur between the inner needle 60 and the outer tube 61. When the backflow detection unit 35 detects this state, it can be determined that the puncture device 41 is in a state immediately before reaching the blood vessel 100. That is, when the backflow detection unit 35 detects backflow into the proximal end side of the inner needle 60 but does not detect backflow between the inner needle 60 and the outer tube 61, the control unit determines that the inner needle 60 has punctured the blood vessel and communicates with the interior of the blood vessel while the outer tube 61 has not punctured the blood vessel and is not in communication with the interior of the blood vessel.

As illustrated in FIG. 16b, in a state in which only the inner needle 60 protruding from the outer tube 61 has penetrated the blood vessel 100 but the outer tube 61 is located in the blood vessel 100, backflow occurs between the inner needle 60 and the outer tube 61, but backflow into the proximal end side of the inner needle 60 does not occur. When the backflow detection unit 35 detects this state, therefore, it can be determined that the puncture device 41 is in a state immediately before penetrating the blood vessel 100. That is, when the backflow detection unit 35 detects backflow between the inner needle 60 and the outer tube 61 but does not detects backflow into the proximal end side of the inner needle 60, the control unit determines that the inner needle 60 has punctured the opposite side of the blood vessel while the outer tube 61 has not yet punctured the opposite side of the blood vessel.

When the backflow detection unit 35 is capable of detecting both the backflow between the inner needle 60 and the outer tube 61 and the backflow into the proximal end side of the inner needle 60, accuracy of the detection of the position of the puncture device 41 can be further improved.

The puncture drive unit 21 only needs to be able to perform puncture by driving the puncture device 41, and is not limited to the configuration of the puncture drive unit 21 in the present embodiment. As illustrated in FIG. 17, the vascular puncture apparatus 12 includes a puncture drive unit 82 including a robot arm 91 capable of moving the puncture device 83 in three dimensions and a probe body 20. The puncture drive unit 82 can cause the puncture device 83 to perform puncture from any position at any angle through control based on a sensor that is not illustrated.

When the arm H is inserted into a base 90 and fixed by a fixing portion 92, the probe body 20 obtains a cross-sectional image, and the control unit 30 determines a puncture timing and puncture parameters from the cross-sectional image. The puncture drive unit 82 punctures the arm H with the puncture device 83 in accordance with a result of the determination whether to puncture, the position or direction of puncture, and the puncture parameters, and detects backflow to control the puncture and the inhibition of backflow.

As described above, (1) a backflow inhibition apparatus according to the present embodiment inhibits an amount of backflow caused by puncturing a living body using a puncture device 41. The backflow inhibition apparatus may include a backflow detection unit 35 that detects an amount or a rate of backflow from a blood vessel to a proximal end side of the puncture device 41, a backflow inhibition unit 36 that operates in such a way as to inhibit the amount of backflow into the puncture device 41, and a control unit 30 that controls operation of the backflow inhibition unit 36 on a basis of the amount or rate of backflow detected by the backflow detection unit 35, in which the control unit 30 operates the backflow inhibition unit 36 in such a way as to inhibit the amount of backflow if the amount or rate of backflow detected by the backflow detection unit 35 is larger than a first threshold. Since the backflow inhibition apparatus configured as described above compares the amount or rate of backflow detected by the backflow detection unit 35 with the threshold and then operates the backflow inhibition unit 36 in such a way as to inhibit the amount of backflow if the amount of backflow is large, it is possible to inhibit an outflow of the backflow to reduce a burden on a body and to keep the outflow from becoming an obstacle when treatment proceeds.
(2) The backflow inhibition apparatus according to (1) may further include a probe body 20 including an imaging unit 22 that obtains a cross-sectional image of a human body, in which the control unit 30 may calculate an estimated insertion depth of the puncture device 41 on a basis of the cross-sectional image obtained by the imaging unit 22, and operate, when an actual insertion depth of the puncture device 41 reaches the estimated insertion depth and the amount or rate of backflow detected by the backflow detection unit 35 is larger than the first threshold, the backflow inhibition unit 36 in such a way as to inhibit the amount of backflow. As a result, since the backflow inhibition apparatus can insert the puncture device 41 deeper into the blood vessel than in a state immediately after the puncture device 41 penetrates a vessel wall, it is possible to perform reliable puncture.
(3) In the backflow inhibition apparatus according to (1) or (2), the control unit 30 may operate the backflow inhibition unit 36 and then detects the amount or rate of backflow using the backflow detection unit 35, and control the backflow inhibition unit 36 on a basis of a result of comparison between the detected amount or rate of backflow and the amount or rate of backflow detected before the backflow inhibition unit 36 is operated. As a result, the backflow inhibition apparatus can confirm whether the backflow inhibition unit 36 is sufficiently functioning, and perform reliable inhibition of backflow.
(4) In the backflow inhibition apparatus according to (1) or (2), the control unit 30 may calculate a cumulative amount of backflow after the backflow detection unit 35 first detects the backflow, and operate, when the calculated cumulative amount of backflow becomes larger than a second threshold, the backflow inhibition unit 36 in such a way as to inhibit the amount of backflow. As a result, since the inhibition of backflow is performed when the cumulative amount of backflow is large, the burden on the body can be further reduced.
(5) The backflow inhibition apparatus according to (1) or (2) may further include a probe body 20 including an imaging unit 22 that obtains a cross-sectional image of a human body, in which the control unit 30 may calculate a cumulative amount of backflow after the backflow detection unit 35 first detects the backflow, calculate an estimated insertion depth of the puncture device 41 on a basis of the cross-sectional image obtained by the imaging unit 22, and operate, when an actual insertion depth of the puncture device 41 reaches the estimated insertion depth and the calculated cumulative amount of backflow is larger than the second threshold, the backflow inhibition unit 36 in such a way as to inhibit the amount of backflow. As a result, since the inhibition of backflow is performed when the cumulative amount of backflow is large, the burden on the body can be further reduced.
(6) In the backflow inhibition apparatus according to (1) or (2), the control unit 30 may measure time elapsed since the backflow detection unit 35 first detected the backflow, and operate, when, after the puncture device 41 punctured the blood vessel to stop insertion, a certain period of time has elapsed since the backflow detection unit 35 first detected the backflow, the backflow inhibition unit 36 in such a way as to inhibit the amount of backflow. As a result, since the inhibition of backflow is performed when the certain period of time has elapsed since the backflow was caused, the burden on the body can be further reduced.
(7) In the backflow inhibition apparatus according to any one of (1) to (6), the puncture device 41 may include, at a proximal end of a needle, a hub 62 having a lumen 62b communicating with the needle, and the backflow inhibition unit 36 may include a valve body 64 having an opening 64a disposed in the lumen 62b of the hub 62 and a pressing body 65a that blocks the opening 64a by pressing the valve body 64. As a result, the valve body 64 is blocked by the movement of the pressing body 65a, and the blood backflow can be effectively inhibited.
(8) In the backflow inhibition apparatus according to any one of (1) to (6), the puncture device 41 may include, at a proximal end of a needle, a hub 62 having a lumen 62b communicating with the needle, and the backflow inhibition unit 36 may include a pressing body 67 disposed adjacent to the lumen 62b of the hub 62 and a pressing drive body 68 that moves the pressing body 67 toward the lumen 62b to collapse the lumen 62b. As a result, the pressing drive body 68 moves the pressing body 67 to block the lumen 62b of the hub 62, and it is possible to effectively inhibit backflow.
(9) In the backflow inhibition apparatus according to any one of (1) to (6), the puncture device 41 may include, at a proximal end of a needle, a hub 62 having a lumen 62b communicating with the needle, and the backflow inhibition unit 36 may include a guide wire drive unit 71 that inserts a guide wire 80 into the lumen 62b of the hub 62. As a result, since the guide wire 80 blocks the lumen 62b of the hub 62, it is possible to effectively inhibit backflow.

The invention here is not limited to the embodiments described above, and those skilled in the art can make various modifications within the technical idea of the invention. For example, although a monitor that displays an obtained cross-sectional image is not illustrated in the present embodiment, the vascular puncture apparatus 10 may be connected to the monitor so that the cross-sectional image can be viewed.

The vascular puncture apparatus 10, 12 may be configured such that the puncture drive unit 21, 82 the backflow detection unit 35, and the control unit 30 are wirelessly connected to one another, and information can be transmitted and received.

The detailed description above describes embodiments of a backflow inhibition apparatus and vascular puncture apparatus representing examples of the new backflow inhibition apparatus and vascular puncture apparatus catheter disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

### REFERENCE SIGNS LIST

10 Vascular puncture apparatus
20 Probe body
21 Puncture drive unit
22 Imaging unit
23 Notification unit
30 Control unit
32 Transmission unit
34 Reception unit
35 Backflow detection unit
36 Backflow inhibition unit
40 Main body
41 Puncture device
60 Inner needle
61 Outer tube
62 Hub
62a Insertion portion
62b Lumen
62c Tube
64 Valve body
64a Opening
65 Cap
65a Pressing body
66 Rotation drive unit
67 Pressing body
68 Pressing drive body
68a Inclined surface
69 Pressing drive portion
70 Guide wire drive device
71 Guide wire drive unit
72 Guide wire insertion unit
73 Coupling unit
80 Guide wire
81 Holder tube

What is claimed is:

1. A vascular puncture apparatus for puncturing a living body, the vascular puncture apparatus comprising:
a main body positionable adjacent the living body;
a puncture device held by the main body and movable towards the living body to cause a distal end of the puncture device to puncture the living body and enter a blood vessel in the living body, the puncture device comprising a hollow needle and a hub on a proximal end side of the hollow needle;
a camera or sensor provided on the puncture device to detect blood backflow from the blood vessel after the puncture device punctures the living body;
a backflow inhibition unit configured to inhibit an amount of the blood backflow into the puncture device;
a control unit operatively connected to: i) the camera or sensor provided on the puncture device to receive information from the camera or about the amount or rate of the blood backflow from the blood vessel after the puncture device punctures the living body; and ii) the backflow inhibition unit to control operation of the backflow inhibition unit based on the information received from the camera or sensor about the amount or rate of the blood backflow detected by the backflow detection unit; and
the control unit being configured to control operation of the backflow inhibition unit to inhibit the amount of the blood backflow if the amount or rate of the blood backflow detected by the backflow detection unit is larger than a threshold.

2. A vascular puncture apparatus according to claim 1, further comprising a transducer provided on the main body and configured to be brought into contact with a surface of the living body to generate an ultrasonic wave and produce a cross-sectional image of an inside of the living body, inclusive of the blood vessel, the control unit being operatively connected to the transducer to receive the cross-sectional image from the transducer and detect a position of the blood vessel in the living body from the cross-sectional image.

3. A vascular puncture apparatus according to claim 1, further comprising a notification unit operatively connected to the control unit to provide notifications about operation of the vasculature puncture apparatus, the notification unit being a display or a sound generator.

4. A vascular puncture apparatus according to claim 1, further comprising a guide wire positioned in a holder tube, the holder tube being connected to a coupling unit that is engageable with an insertion portion of the hub that is on the proximal end side of the hollow needle to allow the guide wire to be introduced into the hollow needle.

5. A vascular puncture apparatus according to claim 1, wherein the backflow inhibition unit comprises a valve positioned in the puncture device, the valve being configured to reduce or stop the blood backflow flow through the puncture device.

6. A vascular puncture apparatus according to claim 1, wherein the backflow inhibition unit further comprises a cap that rotatably engages the hub so that rotation of the cap relative to the hub results in a force applied to the valve to reduce a size of an opening passing axially through the valve.

7. A vascular puncture apparatus according to claim 1, wherein the backflow inhibition unit comprises a pressing body positioned adjacent a lumen in the puncture device and a pressing drive having an inclined surface in contact with the pressing body and configured so that axial movement of the pressing drive urges the pressing body to collapse the lumen.

8. A backflow inhibition apparatus that inhibits an amount of backflow caused by puncturing a living body using a puncture device, the backflow inhibition apparatus comprising:
a backflow detection unit configured to detect an amount or a rate of backflow from a blood vessel to a proximal end side of the puncture device;
a backflow inhibition unit configured to inhibit the amount of backflow into the puncture device;
a control unit configured to control operation of the backflow inhibition unit on a basis of the amount or rate of backflow detected by the backflow detection unit; and
the control unit being configured to operate the backflow inhibition unit to inhibit the amount of backflow if the amount or rate of backflow detected by the backflow detection unit is larger than a first threshold.

9. The backflow inhibition apparatus according to claim 8, further comprising:
a probe body including an imaging unit that obtains a cross-sectional image of a portion of the living body,
the control unit being configured to: i) calculate an estimated insertion depth of the puncture device based on the cross-sectional image obtained by the imaging unit; and ii) operate, when an actual insertion depth of the puncture device reaches the estimated insertion depth and the amount or rate of backflow detected by the backflow detection unit is larger than the first threshold, the backflow inhibition unit in such a way as to inhibit the amount of backflow.

10. The backflow inhibition apparatus according to claim 8, wherein the control unit is configured to: i) operate the backflow inhibition unit and then detect the amount or rate of backflow using the backflow detection unit; and ii) control the backflow inhibition unit based on a result of comparison between the detected amount or rate of backflow and the amount or rate of backflow detected before the backflow inhibition unit is operated.

11. The backflow inhibition apparatus according to claim 8, wherein the control unit is configured to: i) calculate a cumulative amount of backflow after the backflow detection unit first detects the backflow; and ii) operate, when the calculated cumulative amount of backflow becomes larger than a second threshold, the backflow inhibition unit to inhibit the amount of backflow.

12. The backflow inhibition apparatus according to claim 8, further comprising:
a probe body including an imaging unit that obtains a cross-sectional image of a human body, and
wherein the control unit is configured to: i) calculate a cumulative amount of backflow after the backflow detection unit first detects the backflow; ii) calculate an estimated insertion depth of the puncture device based on the cross-sectional image obtained by the imaging unit' and iii) operate, when an actual insertion depth of the puncture device reaches the estimated insertion depth and the calculated cumulative amount of backflow is larger than the second threshold, the backflow inhibition unit in such a way as to inhibit the amount of backflow.

13. The backflow inhibition apparatus according to claim 8, wherein the control unit is configured to: i) measure time elapsed since the backflow detection unit first detected the backflow; and ii) operate after the puncture device punctured the blood vessel to stop insertion and when a certain period of time has elapsed since the backflow detection unit first detected the backflow, the backflow inhibition unit in such a way as to inhibit the amount of backflow.

14. The backflow inhibition apparatus according to claim 8, wherein the puncture device includes, at a proximal end of a needle, a hub having a lumen communicating with the needle, and the backflow inhibition unit includes a valve body having an opening disposed in the lumen of the hub and a pressing body that blocks the opening by pressing the valve body.

15. The backflow inhibition apparatus according to claim 8, wherein the puncture device includes, at a proximal end of a needle, a hub having a lumen communicating with the needle, and the backflow inhibition unit includes a pressing body disposed adjacent to the lumen of the hub and a pressing drive body that moves the pressing body toward the lumen to collapse the lumen.

16. The backflow inhibition apparatus according to claim 8, wherein the puncture device includes, at a proximal end of a needle, a hub having a lumen communicating with the needle, and the backflow inhibition unit includes a guide wire drive unit that inserts a guide wire into the lumen of the hub.

17. A method comprising:
advancing a puncture device toward a living body to puncture the living body with the puncture device and cause a distal end of the puncture device to enter a blood vessel in the living body, the distal end of the puncture device entering the blood vessel in the living body resulting in blood backflow from the blood vessel to a proximal end side of the puncture device;
detecting an amount or rate of the blood backflow from the blood vessel to the proximal end side of the puncture device; and
reducing or stopping the amount or rate of the blood backflow when the amount or rate of blood backflow exceeds a threshold.

18. The method according to claim 17, further comprising:
obtaining a cross-sectional image of a portion of the living body that includes the blood vessel;
calculating an estimated insertion depth of the puncture device based on the cross-sectional image; and
determining when an actual insertion depth of the puncture device reaches the estimated insertion depth; and
reducing or stopping the amount or rate of the blood backflow when: i) the amount or rate of blood backflow exceeds the threshold; and ii) when the actual insertion depth of the puncture device reaches the estimated insertion depth.

19. The method according to claim 17, further comprising stopping the advancing of the puncture device when the amount or rate of the blood backflow exceeds the threshold.

20. The method according to claim 17, further comprising obtaining a cross-sectional image of the blood vessel when the amount or rate of the blood backflow is less than the threshold, using the cross-sectional image of the blood vessel to determine of the blood vessel is an artery and stopping the advancing of the puncture device when the blood vessel is determined to not be an artery.
